(19) United States
(12) Patent Application Publication
(10) Pub. No.: US 2009/0069586 A1
(43) Pub. Date: Mar. 12, 2009

(54) METHOD FOR PRODUCING FATTY ACID ALKYL ESTERS AND/OR GLYCERIN

(75) Inventors: Tomoharu Oku, Abiko-shi (JP); Toshimitsu Moriguchi, Toride-shi (JP); Takeo Akatsuka, Ibaraki (JP); Masanori Nonoguchi, Tsukuba-shi (JP)

Correspondence Address: CONNOLLY BOVE LODGE & HUTZ LLP, 1875 EYE STREET, N.W., SUITE 1100, WASHINGTON, DC, 20006, US

(73) Assignees: Nippon Shokubai Co., Ltd., Osaka (JP); Research Institute of Innovative Technology For the Earth, Kyoto (JP)

(21) Appl. No.: 11/816,754

(22) PCT Filed: Feb. 20, 2006
(86) PCT No.: PCT/JP2006/303512

§ 371 Date: Oct. 22, 2008

(30) Foreign Application Priority Data
Feb. 21, 2005 (JP) 2005-043619
Feb. 21, 2005 (JP) 2005-043620
Feb. 21, 2005 (JP) 2005-043621
Dec. 21, 2005 (JP) 2005-368602

(51) Int. Cl. C11C 1/02; C07C 31/22; C01G 23/04; C01G 25/02; C01B 33/00

(52) U.S. Cl. 554/170; 568/852; 423/598; 423/594.12; 423/344

(57) ABSTRACT

To provide: a production method using a catalyst which can substantially suppress leaching of active metal components and exhibit high activity for both reactions of transesterification of glycerides and esterification of free fatty acids each contained in a fat or oil; and the catalyst. A method for producing fatty acid alkyl esters and/or glycerin comprising a step of bringing a fat or oil into contact with an alcohol in the presence of a catalyst, wherein the catalyst is at least one catalyst selected from the group consisting of the following (I) to (V):
(I) a metal oxide having an ilmenite structure and/or a slyrankite structure;
(II) an oxide containing a metallic element belonging to the Group 12 and a metallic element belonging to the Group 4;
(III) a mixed oxide containing a metallic element belonging to the Group 12 and a tetravalent transition metallic element;
(IV) a metal oxide containing zirconium and at least one metallic element selected from the group consisting of metallic elements belonging to the Groups 4, 5 and 8; and
(V) a metal oxide containing anatase type titanium oxide and/or rutile type titanium oxide, and the metal oxide containing a sulfur component of 700 ppm or less.

### TECHNICAL FIELD

The present invention relates to a method for producing fatty acid alkyl esters and/or glycerin from fats or oils of animal or plant origin. More specifically, the present invention relates to: a method for producing fatty acid alkyl esters and/or glycerin useful for fuel, food, cosmetics, pharmaceuticals and the like purposes; and a catalyst used in the method.

### BACKGROUND ART

Fatty acid alkyl esters derived from vegetable fats and oils are used as cooking oil and, in addition, used in such fields as cosmetics and pharmaceuticals. In recent years, attention has been paid to uses as additives to fuels such as fossil diesel fuel. For example, fatty acid alkyl esters are added to light oil in an amount of several percent as vegetable-derived biodiesel fuel for reducing emission of CO₂. Glycerin is mainly used as a raw material for nitroglycerin and is further used as a raw material for alkyd resins, or for pharmaceuticals, foods, printing inks, cosmetics and the like.

As a method for producing such fatty acid alkyl esters and/or glycerin, it is known transesterification of alcohols with triglyceride, which is a main component of fats and oils. In carrying out such a production method on a commercial scale, a homogenous alkali catalyst is generally used. However, the method needs complicated steps in order to separate and remove the catalyst from the reaction system. Such steps should have been simplified or omitted in order to produce fatty acid alkyl esters and glycerin more effectively. Also, the alkali catalyst causes saponification of free fatty acids contained in the fats and oils. Therefore, soaps are produced as a byproduct, which needs a step of washing the produced soaps with large amounts of water. In addition, emulsification of the soaps would lead to yield decrease of fatty acid alkyl esters and make a subsequent glycerin purification process complicated. Therefore, such problems should have been solved in order to produce fatty acid alkyl esters and glycerin more simply and effectively.

As methods for producing fatty acid alkyl esters, for example, Japanese Kokai Publication No. Hei-07-173103 discloses a method of transesterification of carboxylate, in which a catalyst containing at least one of silicates among the Group 4A (the Group 4) elements in the periodic table as an active component is used, and the silicates may be either crystalline silicates or amorphous silicates. However, this production method should have been improved in catalyst reactivity and catalyst life, as well as reaction yield or conversion rate in the production steps in order to be more useful industrial method to produce fatty acid alkyl esters more efficiently and simply.

As methods for producing lower alkyl esters of fatty acids, for example, Japanese Kokai Publication No. 2000-44984 discloses a method, in which a solid basic catalyst containing a potassium compound and iron oxide, or a potassium compound and zirconium oxide is used as a catalyst. However, this production method should have been improved in reaction yield or conversion rate in the production steps by more sufficient suppression of elution of active material components, whereby to simplify or omit a complicated step, such as removal of the eluted components.

Furthermore, as methods for producing aliphatic esters, for example, U.S. Pat. No. 6,147,196 and French Patent Publication No. 2794768 each disclose a method comprising the following 3 steps: (a) alcoholysis of an oil in the presence of a heterogeneous catalyst and then eliminating an alcohol and separating out glycerin to produce a crude ester containing a monoglyceride; (b) transesterification of the monoglyceride and a fatty acid alkyl ester in the presence of a heterogeneous catalyst to produce a triglyceride and a diglyceride; and (c) recycling the alcohol recovered in the step (a), in which the catalyst of ZnAl₂O₄, xZnO, or yAl₂O₃ (x and y being 0 to 2) is used. However, these production methods also should have been improved to be more efficient and simple production methods of fatty acid alkyl esters. Furthermore, it is well known that titanium (IV) alkoxide is useful as a homogeneous catalyst. However, titanium (IV) oxide usually exhibits low activity (for example, referring to Comparative Examples in Table 3 on page 5 of Japanese Kokai Publication No. Hei-07-173103) Therefore the reaction yield and the conversion rate in the production steps should have been improved.

As methods for producing aliphatic esters, for example, U.S. Pat. No. 5,908,946 discloses a method comprising the following 5 steps: (a) alcoholysis of an oil with a heterogeneous catalyst; (b) evaporating excess alcohol from a reaction mixture after the alcoholysis; (c) separating a mixed solution of a fatty acid alkyl ester and an unreacted oil from glycerin to give the mixed solution of the fatty acid alkyl ester and the unreacted oil; (d) alcoholysis of the mixed solution again with the alcohol recovered in the step (b) in the presence of a heterogeneous catalyst; and (e) re-evaporating excess alcohol from the reaction mixture after the alcoholysis, and separating the glycerin and the fatty acid ester to produce the fatty acid ester, in which catalyst having a spinel structure of ZnAl₂O₄, xZnO, or yAl₂O₃ (x and y being 0 to 2) is preferably used.

With respect to a catalyst containing zinc silicate, for example, British Patent No. 795573 discloses a method, in which animal and vegetable fats and oils are reacted with excess alcohol, and zinc silicate is used as a catalyst.

And with respect to a catalyst containing zinc aluminate, for example, French Patent No. 2752242 discloses a catalyst used in a production of fatty acid alkyl esters and glycerin, the catalyst containing ZnAl₂O₄, xZnO, or yAl₂O₃ (x and y being 0 to 2). In this production method, ZnAl₂O₄, xZnO, and yAl₂O₃ each containing a spinel structure are preferably used.

For example, French Patent No. 2772756 discloses a catalyst used in a reaction of a plant or animal oil with an excess of alcohol, the catalyst being either aluminates or silicates each based on zinc, titanium, or tin. Also disclosed is a catalyst based on zinc, tin, or titanium, and the catalyst being, for example, aluminates or silicates, preferably aluminates. Alumina-carried titania is illustrated as a catalyst, and zinc aluminate is mainly used as a catalyst in Examples. And for example, Japanese Kokai Publication No. 2005-200398 discloses a method for producing fatty acid alkyl esters and/or glycerin comprising a step of bringing a fat or oil into contact with an alcohol in the presence of a catalyst, wherein the catalyst contains at least one kind of a metal element selected from the group consisting of group 4 and 5 metal elements. However, if such catalysts are used in a reaction mixture containing a free fatty acid or water, leaching of metal components is observed due to soaps as a by production, and repeated reactions. Thereby, the separation and purification step becomes complicated and the catalyst life is shortened. Therefore, there is room for improvement in the catalysts.

### SUMMARY OF THE INVENTION

The present invention has been made in view of the above state of the art. That is, a catalyst used in the present invention can substantially suppress leaching of active metal components and exhibit high activity for both reactions of transesterification of glycerides and esterification of free fatty acids each contained in a fat or oil. Thereby, the present invention has an object to provide: an efficient and selective production method of fatty acid alkyl esters and/or glycerin suitable for applications, such as food and fuel, which can simplify or omit a complicated step, such as removal step of catalysts; and a catalyst used in the production method.

The present inventors have made various investigations about methods for producing fatty acid alkyl esters and/or glycerin. And they have noted that a method for producing fatty acid alkyl esters and/or glycerin by bringing a fat or oil into contact with alcohols in the presence of a catalyst is industrially useful. They have found that, in such steps, a metal oxide having an ilmenite structure and/or a slyrankite structure has an ability of catalyzing esterification and transesterification simultaneously, and is unaffected by a mineral acid or a metal component contained in a fat or oil and, further avoids decomposition of alcohols. Also, they have found that leaching of active metal components is sufficiently suppressed, and whereby catalyst activity and catalyst life are sufficiently improved because the catalyst has a specific crystal structure. And they also have found that the catalyst can remarkably simplify or omit the catalyst separation and removal step and a catalyst recovery step, as compared with homogeneous catalysts used in conventional methods, and can be repeatedly used for the reaction. Therefore, they have resolved the above-mentioned problems. And they have found that if such a metal oxide having an ilmenite structure contains titanium and at least one metallic element selected from the group consisting of metallic elements belonging to the Groups 7, 8, 9, 10, and 12, the catalyst life is more improved and leaching of the active metal components is more sufficiently suppressed, which can exhibit effects of the present invention, and found that MnTiO₃, CoTiO₃, ZnTiO₃, FeTiO₃, and NiTiO₃ each containing an ilmenite structure are especially useful.

The present inventors also have found that an oxide catalyst containing a metallic element belonging to the Group 12 and a metallic element belonging to the Group 4, or a mixed oxide containing a metallic element belonging to the Group 12 and a tetravalent transition metallic element has an ability of catalyzing esterification and transesterification simultaneously, and is unaffected by a mineral acid or a metal component contained in a fat or oil and, further, avoids decomposition of an alcohol. And they also have found that particularly if an acid metal such as titanium or zirconium is used as the metallic element belonging to the Group 4 or the tetravalent metallic element, and a basic metal such as zinc is used as the metallic element belonging to the Group 12, and then the acid metal and the basic metal are mixed, zinc can be stably fixed. They also have found that if these metal oxides are crystallized, they can be more stable catalysts. For example, Zn₂TiO₄ with a spinel structure has thermal stability higher than that of mixed oxide ZnO/TiO₂ especially amorphous ZnO/TiO₂, and can be more stabilized. And in ZnZrO₃ having a cubic structure, both zinc and zirconium can be stabilized. And they also have found that the catalyst can remarkably simplify or omit the catalyst recovery step, as compared with homogeneous catalysts used in conventional methods and can be repeatedly used for the reaction, and the catalyst can make reaction yield and conversion rate more improved, because the catalysts suppress leaching of the active metal components and have long catalyst life. Therefore, they have resolved the above-mentioned problems. They also have found that even if the reaction mixture contains a free fatty acid and water, no soap is generated as a byproduct, and the catalyst is allowed to have long life, which can reduce catalyst-related costs, and permit stable and long-term production of fatty acid alkyl esters and/or glycerin.

They have also found that, in the present invention, a metal oxide containing zirconium and at least one metallic element selected from the group consisting of metallic elements belonging to the Groups 4, 5, and 8 as essential components has an ability of catalyzing esterification and transesterification simultaneously, and is unaffected by a mineral acid or a metal component contained in a fat or oil and, further, avoids decomposition of an alcohol. Also, they have found that high activity and immobilization of active metal component in solid catalyst to a raw material or a product can be sufficiently attained by the catalyst because the catalyst essentially contains zirconium. And they also have found that the catalyst can remarkably simplify or omit the catalyst recovery step, as compared with homogeneous catalysts used in conventional methods and can be repeatedly used for the reaction, and make reaction yield and conversion rate more improved. Therefore, they have resolved the above-mentioned problems. And they have found that if such a catalyst contains zirconium oxide having a monoclinic structure, the catalyst reactivity and the catalyst life are more improved and leaching of the active metal components is more sufficiently suppressed, which can exhibit effects of the present invention.

They have found that, in the present invention, a catalyst containing anatase type titanium oxide and/or rutile type titanium oxide has an ability of catalyzing esterification and transesterification simultaneously, and is unaffected by a mineral acid or a metal component contained in a fat or oil and, further, avoids decomposition of alcohols. Also, they have found that use of such crystalline titanium oxide can sufficiently suppress leaching of titanium component, which is an active component. They also have found that if the catalyst is used after being adjusted such that concentration of the sulfur components contained in the catalyst has a specific value or less, for example, by reducing residual sulfur components, the activity of titanium oxide whose low activity has made practical application difficult so far can be improved, and the catalyst life can be extended. And they also have found that the catalyst can remarkably simplify or omit the catalyst separation and removal step and the catalyst recovery step, as compared with homogeneous catalysts used in conventional methods, and can be repeatedly used for the reaction. Therefore, they have resolved the above-mentioned problems. And they have found that if the catalyst contains an oxide of at least one metallic element selected from the group consisting of metallic elements belonging to the Groups 4, 13, and 14, the catalyst life and the catalyst activity are more improved and leaching of the active metal components is more sufficiently suppressed, which can exhibit effects of the present invention. Thereby, the present invention has been completed. Therefore, the present invention has been completed.

That is, the present invention relates to a method for producing fatty acid alkyl esters and/or glycerin comprising a step of bringing a fat or oil into contact with an alcohol in the presence of a catalyst,

wherein the catalyst is at least one catalyst selected from the group consisting of the following (I) to (V):

(I) a metal oxide having an ilmenite structure and/or a slyrankite structure;
(II) an oxide containing a metallic element belonging to the Group 12 and a metallic element belonging to the Group 4;
(III) a mixed oxide containing a metallic element belonging to the Group 12 and a tetravalent transition metallic element;
(IV) a metal oxide containing zirconium and at least one metallic element selected from the group consisting of metallic elements belonging to the Groups 4, 5 and 8; and
(V) a metal oxide containing anatase type titanium oxide and/or rutile type titanium oxide, and the metal oxide containing a sulfur component of 700 ppm or less.

### DETAILED DESCRIPTION OF THE INVENTION

The present invention will, hereinafter, be described in more detail.

In the method for producing fatty acid alkyl esters and/or glycerin according to the present invention, a fat or oil, and an alcohol are contacted with each other in the presence of at least one catalyst selected from the group consisting of (I) to (V). Such a catalyst may be at least one catalyst among the above-mentioned catalysts (I) to (V). Any of the catalysts can sufficiently exhibit functional effects of the present invention. The catalysts (I) to (V) will, hereinafter, be described.

If the catalyst (I) is used, a fat or oil, and an alcohol are contacted with each other in the presence of a metal oxide having an ilmenite structure and/or a slyrankite structure. In such a production method, a reaction can be performed with high efficiency because a metal oxide having an ilmenite structure and/or a slyrankite structure, which is used as a catalyst, sufficiently suppresses leaching of the active metal components and improves catalyst activity and catalyst life.

The above-mentioned metal oxide is not especially limited as long as it has an ilmenite structure and/or a slyrankite structure, and may be, for example, a mixture of a single oxide or a mixed oxide, or contain an active component (for example, a single or mixed oxide of metallic element) carried or immobilized on a carrier. Examples of the carrier on which the active component is carried or fixed include silica, alumina, silica-alumina, various zeolites, activated carbon, kieselguhr, zirconium oxide, rutile type titanium oxide, tin oxide, lead oxide. As mentioned above, preferred embodiment of the present invention includes a method for producing fatty acid alkyl esters and/or glycerin comprising a step of bringing a fat or oil into contact with an alcohol in the presence of a catalyst,

wherein the catalyst is a metal oxide having an ilmenite structure and/or a slyrankite structure.

The above-mentioned ilmenite structure is expressed by the formula ABX₃ (A and B being cations and X being an anion), and means a rhombohedral lattice which is a slightly distorted hexagonal closest packing of X with the octahedral holes occupied by A and B regularly arranging as 6 coordination. For example, a mixed oxide represented by FeTiO₃ has a structure in which a position of Al of α-alumina (corundum type) is replaced with Fe and Ti regularly. If the metal oxide having such a structure is used as the catalyst, the catalyst has sufficient insolubility to any of a fat or oil and an alcohol, which are raw materials of the present invention, and a product (fatty acid alkyl esters, glycerin and the like), and the catalyst life is remarkably improved. Therefore, in the production method of the present invention, recycling efficiency of the catalyst is more improved and a catalyst separation and removal step can be remarkably simplified or omitted. Therefore, utility costs and equipment costs are sufficiently reduced.

It is preferable that the metal oxide has such an ilmenite structure in which at least one of A and B is titanium in the above formula. For example, MnTiO₃, CoTiO₃, ZnTiO₃ (ilmenite type), FeTiO₃, and NiTiO₃ may be mentioned. Among them, the catalyst contains a metal oxide having an ilmenite structure containing titanium and at least one metallic element selected from the group consisting of metallic elements belonging to the Groups 7, 8, 9, 10, and 12. The catalyst containing such a metal oxide is part of preferable embodiment of the present invention. In this case, leaching of the active component is more sufficiently suppressed, which permits sufficient exhibition of functional effects of the present invention. More preferably, the metal oxide is MnTiO₃, CoTiO₃, ZnTiO₃ (ilmenite type), FeTiO₃, and NiTiO₃.

The slyrankite structure means a mixed oxide and/or solid solution, mainly composed of titanium and zirconium, which has the same structure as in orthorhombic α-PbO₂, represented by Ti_{0.67}Zr_{0.33}O₂ discovered by A. Willgallis et al in 1983. That is, the slyrankite structure characteristically has a structure in which Ti⁴⁺ and Zr⁴⁺ are randomly disposed at Pb⁴⁺ of α-PbO₂. Specifically, the slyrankite structure is a mixed oxide and/or solid solution in which atomic ratio of total atomic number of Ti⁴⁺ and Zr⁴⁺ to oxygen is 1:2. In the present invention, the atomic ratio of Ti to Zr is preferably (1 to 0.2):(0 to 0.8), more preferably (0.8 to 0.3):(0.2 to 0.7), and still more preferably (0.7 to 0.4):(0.3 to 0.6).

The catalyst containing such a metal oxide exhibits activity higher than that of titanium oxide, silica-carried titanium oxide or the like. Therefore, according to the producing method of the present invention, recycling efficiency of the above-mentioned catalyst is improved and thereby utility costs and equipment costs are sufficiently reduced, which permits highly selective production of fatty acid alkyl esters and/or glycerin with high yield. It can be determined using powder X-ray diffraction measurement (XRD) whether the above-mentioned catalyst has an ilmenite structure or a slyrankite structure.

If the above-mentioned catalyst(s) (II) and/or (III) is/are used, a fat or oil, and an alcohol are contacted with each other in the presence of an oxide containing a metallic element belonging to the Group 12 and a metallic element belonging to the Group 4 and/or a mixed oxide containing a metallic element belonging to the Group 12 and a tetravalent transition metallic element. If the catalyst according to the present invention is an oxide containing the above-mentioned metallic elements, the metallic elements can be stably fixed. Therefore, such a catalyst can be preferably used as a catalyst. And the reaction can be more effectively carried out because the catalyst sufficiently suppresses leaching of the active metal components and exhibits high catalyst activity efficiently. As mentioned above, preferred embodiment of the present invention includes a method for producing fatty acid alkyl esters and/or glycerin comprising a step of bringing a fat or oil into contact with an alcohol in the presence of a catalyst,

wherein the catalyst is an oxide containing a metallic element belonging to the Group 12 and a metallic element belonging to the Group 4, and/or

a method for producing fatty acid alkyl esters and/or glycerin comprising a step of bringing a fat or oil into contact with an alcohol in the presence of a catalyst,

wherein the catalyst is a mixed oxide containing a metallic element belonging to the Group 12 and a tetravalent transition metallic element.

As the above-mentioned metallic element belonging to the Group 12, one or more elements among zinc element, cadmium element, and mercury element are preferred. Among them, zinc element is preferred in view of toxicity. Thus, the method for producing fatty acid alkyl esters and/or glycerin, wherein the metallic element belonging to the Group 12 is zinc element is part of preferable embodiment of the present invention.

As the above-mentioned metallic element belonging to the Group 4, one or more elements among titanium element, zirconium element, and hafnium element are preferred.

The above-mentioned tetravalent transition metallic element is not especially limited and may be any transition metallic elements which can be tetravalent. Preferred are one or more elements among titanium element, zirconium element, hafnium element, vanadium element, niobium element, tantalum element, chromium element, molybdenum element, tungsten element, manganese element, rhenium element, iron element, ruthenium element, osmium element, cobalt element, nickel element, palladium element, platinum element, cerium element, and terbium element.

As the above-mentioned metallic element belonging to the Group 4 and the above-mentioned tetravalent transition metallic element, the metallic elements mentioned above can be preferably used, respectively. Among them, titanium element or zirconium element is preferred in both metallic elements, because such an element enables the metallic element belonging to the Group 12 to be fixed stably and enables the tetravalent to exist stably. That is, it is preferable that the catalyst is an oxide containing a metallic element belonging to the Group 12, and Ti or Zr.

The catalyst may be the oxide containing a metallic element belonging to the Group 12 and a metallic element belonging to the Group 4, or the mixed oxide containing a metallic element belonging to the Group 12 and a tetravalent transition metallic element. And the oxide may contain one or more kinds of metallic elements belonging to the Group 12 and/or contain one or more kinds of metallic elements belonging to the Group 4, and the mixed oxide may contain one or more kinds of metallic elements belonging to the Group 12 and/or contain one or more kinds of tetravalent metallic elements. As a ratio of the above-mentioned metallic element belonging to the Group 12 to the metallic element belonging to the Group 4 or to the tetravalent metallic element, it is preferable that an atomic ratio of the metallic element belonging to the Group 12 to the metallic element belonging to the Group 4 or to the tetravalent metallic element is 0.05 or more and 10 or less. If the atomic ratio is less than 0.05, improvement effect of the catalyst activity may be insufficiently exhibited. If the atomic ratio is more than 10, leaching of the active metal components (the metallic element belonging to the Group 12, the metallic element belonging to the Group 4, and the tetravalent transition metallic element) of the catalyst into a reaction mixture may be insufficiently suppressed. Preferably, the atomic ratio is 0.1 or more and 5 or less. More preferably, the atomic ratio is 0.2 or more and 4 or less.

The oxide catalyst containing the metallic element belonging to the Group 12 and the metallic element belonging to the Group 4 is not especially limited as long as the catalyst is an oxide containing the above elements. Such an oxide catalyst is preferably a mixed oxide, a mixture of a single oxide or the like, for example. Among them, such an oxide catalyst is preferably a mixed oxide containing the metallic element belonging to the Group 12 and the metallic element belonging to the Group 4, because the activity as a catalyst is high and the metallic element belonging to the Group 12 can be stably fixed. As mentioned above, the method for producing fatty acid alkyl esters and/or glycerin, wherein the oxide is a mixed oxide is part of preferable embodiment of the present invention.

The above-mentioned mixed oxide may be crystallized or not crystallized, but preferably crystallized. And the crystallized mixed oxide has a structure containing the metallic element belonging to the Group 12, and the metallic element belonging to the Group 4 or the tetravalent transition metallic element in its crystal lattice. If the mixed oxide is crystallized, leaching of the metallic element belonging to the Group 12, and the metallic element belonging to the Group 4 or the tetravalent transition metallic element, which are active metal components, can be sufficiently suppressed, and therefore the catalyst can be more stable and has more improved catalyst activity and life.

A form of the mixed oxide is not especially limited. For example, the following forms may be mentioned: a form, in which the metallic element belonging to the Group 12, and the metallic element belonging to the Group 4 or the tetravalent transition metallic element are bonded by covalent bond with an oxygen atom therebetween; a form, in which the metallic element belonging to the Group 12, and the metallic element belonging to the Group 4 or the tetravalent transition metallic element are bonded, and the bonded elements and an oxygen atom are bonded by covalent bond; and a composite of oxides of the metallic element belonging to the Group 12, and the metallic element belonging to the Group 4 or the tetravalent transition metallic element, and solid solution thereof. A form, in which a mixed oxide, a complex and the like is carried or immobilized on a carrier, may be also mentioned. Examples of the carrier include silica, alumina, silica-alumina, various zeolites, activated carbon, kieselguhr, zirconium oxide, rutile type titanium oxide, tin oxide, and lead oxide.

The oxide contains ZnₓMOₙ (in the formula, M represents Ti and/or Zr; x is a number of 0.05 or more and 10 or less; and n is a number determined such that ZnₓMOₙ is electrically neutral).

The above-mentioned x is preferably 0.05 or more and 10 or less, more preferably 0.1 or more and 5 or less, and most preferably 0.2 or more and 4 or less.

The oxide is preferably ZnZrO₃, ZnTiO₃, Zn₂TiO₄, Zn_{2/3}TiO_{8/3}, or Zn₄TiO₆. It is more preferably ZnZrO₃, ZnTiO₃, Zn₂TiO₄, or Zn_{2/3}TiO_{8/3}, and still more preferably, ZnZrO₃, ZnTiO₃, or Zn₂TiO₄; and most preferably ZnZrO₃ and Zn₂TiO₄. The Zn_{2/3}TiO_{8/3} used herein is synonymous with Zn₂Ti₃O₈. That is, the general formula ZnₓMOₙ includes Zn_{xxy}MyO_{nxy}, in which a ratio of each constituent atom is an integer ratio. Thus, the method for producing fatty acid alkyl esters and/or glycerin, wherein the oxide contains at least one selected from the group consisting of ZnZrO₃, ZnTiO₃, Zn₂TiO₄, Zn_{2/3}TiO_{8/3}, and Zn₄TiO₆, is part of preferable embodiment of the present invention.

It is preferable that the oxide is cubic. The term “cubic” used herein means a crystal system satisfying a=b=C and α=β=γ=90° if crystallographic axes of a unit cell of a crystal are defined as a, b, and c, and the axial angles are defined as α, β, and γ.

If the oxide has such a structure, both of the metallic element belonging to the Group 12, and the metallic element belonging the Group 4 or the tetravalent transition metallic element are stabilized. If oxide has such a structure, the catalyst has sufficient immobility to any of a fat or oil, an alcohol, which are raw materials, and a product (fatty acid alkyl esters, glycerin and the like), and the catalyst life is remarkably improved. Therefore, in the production method of the present invention, recycling efficiency of the catalyst is more improved and a catalyst separation and removal step can be remarkably simplified or omitted. Therefore, utility costs and equipment costs are sufficiently reduced. Examples of the cubic oxide include ZnZrO₃, ZnTiO₃ (spinel type), Zn₂TiO₄, and Zn₂Ti₃O₈.

It is preferable that the oxide has a spinel structure in the cubic structure. The spinel structure is a general term for a mixed oxide represented by the formula AB₂O₄. And the spinel structure means a normal spinel structure that is a cubic closed packed structure of oxide ions with one-eighth of tetrahedral holes regularly occupied by A ions and one-half of the octahedral holes regularly occupied by B ions, or means an inverse spinel structure that is a close-packed cubic lattice of oxide ions with the tetrahedral holes occupied by half of B ions, and the octahedral holes occupied by A ions and the rest of the B ions. The spinel structure may have a defect. A metal oxide having such a structure preferable has titanium as at least one of A and B in the above formula. For example, Zn₂TiO₄, and spinel type ZnTiO₃ are preferred. Among them, a metal oxide having a spinel structure containing titanium and a metallic element belonging to the Group 12 are preferred. In addition, ZnTiO₃ (spinel type) is not distinguished from ZnTiO₃ having an ilmenite structure in view of composition formula, but having a lattice defect, ZnTiO₃ (spinel type) is an oxide having not an ilmenite structure but a spinel structure. The catalyst containing such a metal oxide is part of preferable embodiment of the present invention. If the catalyst has a spinel structure, the catalyst is preferable as a catalyst because it can exhibit higher thermostability and can be more stabilized than the catalyst having an ilmenite structure. Furthermore, leaching of the active component is more sufficiently suppressed, which permits sufficient exhibition of functional effects of the present invention. It is more preferable that the metal oxide is Zn₂Tio₄.

The catalyst containing such a metal oxide exhibits activity higher than that of titanium oxide, silica-carried titanium oxide or the like. Therefore, according to the producing method of the present invention, recycling efficiency of the catalyst is improved and thereby utility costs and equipment costs are sufficiently reduced, which permits highly selective production of fatty acid alkyl esters and/or glycerin with high yield. Powder X-ray diffraction analysis (XRD) can show whether the catalyst is cubic, specifically the catalyst has a spinel structure and the like.

The crystal structure of the catalyst according to the present invention is not especially limited unless functional effects of the present invention are accomplished. And the catalyst may have a structure other than the above-mentioned structure. The catalyst preferably has a rutile structure or a wurtzite structure, for example. Furthermore, the catalyst has a crystal structure not specified. The catalyst having a crystal structure not specified may be, for example, Zn₄TiO₆.

If the catalyst (IV) is used, a fat or oil and an alcohol are contacted with each other in the presence of a metal oxide containing zirconium and at least one metallic element selected from the group consisting of metallic elements belonging to the Groups 4, 5 and 8 as an essential component. In such a production method, a reaction can be performed with high efficiency because a metal oxide (hereinafter, referred to also as “metal oxide catalyst”) containing zirconium and the above-mentioned metallic element, which is used as a catalyst, sufficiently suppresses leaching of active metal components and exhibits high catalyst activity. Thus, preferred embodiment of the present invention includes a method for producing fatty acid alkyl esters and/or glycerin comprising a step of bringing a fat or oil into contact with an alcohol in the presence of a catalyst,

wherein the catalyst is a metal oxide containing zirconium and at least one metallic element selected from the group consisting of metallic elements belonging to the Groups 4, 5 and 8.

The above-mentioned metal oxide is not especially limited as long as it contains the above-mentioned essential components, and may be, for example, a mixture of a single oxide or a mixed oxide, or contain an active component carried or immobilized on a carrier. And the metal oxide may be also in the form, in which one of the above-mentioned essential components is used as the carrier and the other component is carried or immobilized as the active component. Such an embodiment is part of preferable embodiment.

Examples of the carrier on which the active component is carried or immobilized include silica, alumina, silica-alumina, various zeolites, activated carbon, kieselguhr, zirconium oxide, rutile type titanium oxide, tin oxide, lead oxide. And in particularly, zirconium oxide is preferably used as the carrier. Among them, the above-mentioned metal oxide is preferably in the form, in which zirconium oxide is used as the carrier, and a single or mixed oxide of at least one metallic element selected from the group consisting of metallic elements belonging to the Groups 4, 5 and 8 is used as the active component, and the active component is carried or immobilized on the carrier. Use of zirconium oxide as the carrier permits exhibition of higher activity as compared with cases where a silica carrier is used as a carrier on which oxides of the above-mentioned metallic element, such as titanium oxide, or the above-mentioned active metallic element, such as silica-carried titanium oxide is carried or immobilized, and also extends life of a vanadium catalyst generally having a short life. Therefore, recycling efficiency of the above-mentioned catalyst is improved and thereby utility costs and equipment costs are sufficiently reduced, which permits highly selective production of fatty acid alkyl esters and/or glycerin with high yield.

The above-mentioned zirconium oxide preferably has a monoclinic structure. Such monoclinic zirconium oxide further improves the catalytic activity, and suppresses leaching of the above-mentioned metallic element which is the active metal component. That is, it is part of preferable embodiment that the catalyst contains zirconium oxide having a monoclinic structure as an essential component.

In the above-mentioned metal oxide, the above-mentioned at least one metallic element selected form the group consisting of metallic elements belonging to the Groups 4, 5 and 8 is preferably a single or mixed oxide as mentioned above. The mixed oxide may be a mixed oxide of the at least one metallic element selected form the group consisting of metallic elements belonging to the Groups 4, 5 and 8, and other metallic elements. The single or mixed oxide more preferably contains at least one metallic element selected from the group consisting of Ti, V, and Fe. And it is particularly preferable that Ti is essentially contained. Specific examples of the mixed oxide containing Ti include titanium oxide such as anatase type TiO₂ and rutile type TiO₂, titania silica, titania zirconia, titania magnesia, titania calcia, titania yttria, titania boria, titania-tin oxide; titanium vanadium mixed oxide such as TiVO₄; vanadium oxide; iron vanadium mixed oxide such as FeVO₄; cobalt vanadium mixed oxide such as CO₂V₂O₇; cerium vanadium mixed oxide such as CeVO₄; zinc vanadium mixed oxide, nickel vanadium mixed oxide, copper vanadium mixed oxide, scandium vanadium mixed oxide, yttrium vanadium mixed oxide, lanthanum vanadium mixed oxide, tin vanadium mixed oxide, lead vanadium mixed oxide, antimony vanadium mixed oxide, bismuth vanadium mixed oxide, selenium vanadium mixed oxide, tellurium vanadium mixed oxide; iron oxide. These may be used singly or in combination of two or more species. Among them, anatase type TiO₂, rutile type TiO₂, and iron vanadium mixed oxide are preferred. Rutile type TiO₂ and triclinic FeVO₄ are more preferred.

The mixed oxide may be in any form, and the following forms may be mentioned: a form in which a first atom and a second atom are bonded by covalent bond with an oxygen atom therebetween; a form, in which a first atom and a second atom are bonded, and the bonded atoms and an oxygen atom are bonded by covalent bond; and a composite of an oxide of a first atom and an oxide of a second atom and solid solution thereof.

It is preferable that the catalyst contains a metal oxide having a triclinic structure as an active component. The triclinic structure means a crystal system which has all the three crystallographic axes obliquely crossing with one another, the lengths of the crystallographic axes being different from one another, and has a triclinic lattice. Among them, the metal oxide is preferably an oxide containing iron, vanadium, and zirconium, in which triclinic FeVO₄ mixed oxide is carried or fixed on zirconium oxide.

The above-mentioned oxide containing iron, vanadium, and zirconium may be, other than the above oxide, an oxide, in which iron vanadium mixed oxide, which is not triclinic, is carried or fixed on zirconium oxide, or a ternary mixed oxide containing iron, vanadium, and zirconium. They are part of preferable embodiment of the present invention. Use of the oxide containing iron, vanadium, and further zirconium suppresses leaching of the active component and thereby extends the catalyst life, as compared with common oxides containing iron and vanadium.

If the above-mentioned metal oxide is a mixed oxide of zirconium and the above-mentioned metallic element, the mixed oxide is preferably Zr_{0.5}Tio_{0.5}O₂ (slyrankite). Such a form, in which the above-mentioned catalyst has a Zr_{0.5}Tio_{0.5}O₂ (slyrankite) structure, is part of preferable embodiment of the present invention. The slyrankite structure means the structure as mentioned above. It can be determined using powder X-ray diffraction measurement (XRD) whether the above-mentioned catalyst has monoclinic zirconium oxide, triclinic mixed oxide of iron and vanadium, or Zr_{0.5}Tio_{0.5}O₂ (slyrankite) structure.

The content of the zirconium in the above-mentioned metal oxide has a lower limit of 1% by weight, and an upper limit of 80% by weight as a metal, relative to 100% by weight of the total amount of the catalyst of the present invention. If the content is less than 1% by weight, the metal oxide may neither exhibit high activity nor suppress leaching to a raw material or a product sufficiently. If the content is more than 80% by weight, high activity may not be exhibited any more. More preferably, the lower limit is 2% by weight and the upper limit is 75% by weight.

And the content of the above-mentioned at least one metallic element selected from the group consisting of metallic elements belonging to the Groups 4, 5, and 8 has a lower limit of 1% by weight and an upper limit of 80% by weight as a metal, relative to 100% by weight of the total amount of the catalyst of the present invention. If the content is less than 1% by weight, insufficient catalyst activity may not improve reaction efficiency. If it exceeds 80% by weight, the oxide of the metallic element agglutinates on the carrier, and thereby the metal oxide may not exhibit high activity sufficiently, and exhibition of functional effects attributed to the presence of the zirconium may be insufficient. More preferably, the lower limit is 2% by weight and the upper limit is 75% by weight. The content of the metallic element can be determined by X-ray fluorescence (XRF) analysis.

If the catalyst (V) is used, a fat or oil, and an alcohol are contacted with each other in the presence of a metal oxide containing anatase type titanium oxide and/or rutile type titanium oxide. A reaction can be performed with high efficiency because such a catalyst containing crystalline titanium oxide sufficiently suppresses leaching of the titanium component, which is an active component, and improves the catalyst activity and the catalyst life sufficiently. Thus, preferred embodiment of the present invention includes a method for producing fatty acid alkyl esters and/or glycerin comprising a step of bringing a fat or oil into contact with an alcohol in the presence of a catalyst,

wherein the catalyst is a metal oxide containing anatase type titanium oxide and/or rutile type titanium oxide, and the catalyst contains a sulfur component of 700 ppm or less.

Content of titanium contained in the above-mentioned catalyst suitably has a lower limit of 0.5% by weight, and an upper limit of 60% by weight. The lower limit is more preferably 1% by weight and still more preferably 2% by weight. The upper limit is more preferably 45% by weight, and still more preferably 30% by weight.

Other catalysts may be used in combination with the above-mentioned catalysts (I) to (V) in the production method according to the present invention. The above-mentioned catalyst may be used singly or in combination of two or more of the catalysts. And the catalysts may contain impurities and other components produced in the catalyst preparation step unless effects of the present invention are accomplished.

The terms “anatase structure” and “rutile structure” mean a crystal structure belonging to the tetragonal system and are formed by a compound represented by AB₂ (A: positive atom, B: negative atom). In such crystal structure, A atom is octahedrally coordinated by B atom. In the anatase structure, resulting octahedrons form a skeletal structure as a result of each octahedron sharing 4 edges with each neighboring octahedron. In the rutile structure, resulting octahedrons form a skeletal structure as a result of each octahedron sharing 2 edges with each neighboring octahedron. Such compounds having the rutile structure can be obtained by calcinating compounds having the anatase structure.

Powder X-ray diffraction measurement (XRD) can show whether the catalyst contains the anatase type titanium oxide and/or the rutile type titanium oxide.

The catalyst may contain a single oxide or a mixed oxide other than the anatase type titanium oxide and/or the rutile type titanium oxide. And the catalyst also may contain the above titanium oxide as a carrier or an active component, the active component being carried or immobilized on the carrier. Examples of the carrier include silica, alumina, silica-alumina, various zeolites, activated carbon, kieselguhr, zirconium oxide, tin oxide, lead oxide, other than the above-mentioned titanium oxide.

It is preferably that the above-mentioned catalyst further contains an oxide of at least one metallic element selected from the group consisting of metallic elements belonging to the Groups 4, 13, and 14. For example, it is preferable that the catalyst is a mixture of the oxide and the above-mentioned titanium oxide, or contains the oxide or the above-mentioned titanium oxide as a carrier or an active component, the active component being carried on the carrier.

It is most preferable that the catalyst contains an oxide of at least one metallic element selected from the group consisting of metallic elements belonging to the Groups 4, 13, and 14 as a carrier, and contains anatase type titanium oxide and/or rutile type titanium oxide as an active component. Use of such a catalyst can sufficiently suppress leaching of the titanium component, which is the active component, and improves the catalyst activity and the catalyst life. Therefore, such a catalyst may be most preferably used for the production method of the present invention.

The above-mentioned oxide containing at least one metallic element selected from the group consisting of metallic elements belonging to the Groups 4, 13, and 14 is preferably a single oxide of these metallic element, a mixture thereof, or a mixed oxide of these metallic elements. The mixed oxide may be a mixed oxide of at least one metallic element selected from the group consisting of metallic elements belonging to the Groups 4, 13, and 14, and other metallic elements. The single or mixed oxide of the metallic elements more preferably contains at least one metallic element selected from the group consisting of Si, Zr, and Al. For example, silica, alumina, silica-alumina, and zirconium oxide may be mentioned.

The oxide of at least one metallic element selected from the group consisting of metallic elements belonging to the Groups 4, 13, and 14 may contain amorphous titanium oxide, or a mixed oxide consisted of titanium and a metal other than titanium, but contain no anatase type titanium oxide and/or rutile type titanium oxide.

In the above-mentioned catalyst, content (not including titanium content constituting anatase type titanium oxide and/or rutile type titanium oxide) of the at least one metallic element selected from the group consisting of metallic elements belonging to the Groups 4, 13, and 14 has a lower limit of 1% by weight and an upper limit of 80% by weight in metal conversion, relative to the total amount of the catalyst 100% by weight. Use of the catalyst containing metallic element within the range can further improve the catalyst activity and the reaction efficiency, which allows sufficient exhibition of functional effects of the present invention. More preferably, the lower limit is 2% by weight and the upper limit is 75% by weight. The content of metallic element can be determined by X-ray fluorescence (XRF) analysis.

The catalyst preferably contains a sulfur component of 700 ppm or less. If the catalyst contains a sulfur component of 700 ppm or more, the catalyst activity and the catalyst life might be insufficiently improved. Therefore, functional effects of the invention, in which the catalyst separation, removal, and recovery steps can be remarkably simplified or omitted and the catalyst can be repeatedly used for continuous reactions, might not be exhibited. The content of sulfur components is preferably 500 ppm or less, and more preferably 200 ppm or less. The content of sulfur components in the catalyst can be determined by fluorescence X rays (XRF) analysis or high frequency induction plasma (ICP) emission spectrometry. According to XRF analysis, the component can be determined by measuring a catalyst powder as it is or by a glass bead method. According to ICP emission spectrometry, the component can be determined by measuring a catalyst powder after being dissolved in a hydrofluoric acid aqueous solution. However, the sulfur component is preferably determined by a glass bead method of XRF, in view of ease of measurement.

For reducing the sulfur component contained in the catalyst, for example, the catalyst may be prepared using no sulfate as a raw material, or using sufficiently reduced amount of sulfate, or washed with sufficient amount of a solvent such as water.

A calcined metal oxide may be used as the above-mentioned catalysts (catalysts (I) to (V)), which can further suppress leaching of the active metal component. The calcination temperature is preferably determined in consideration of the catalyst surface area and the crystal structure, and preferably has a lower limit of 280° C. and an upper limit of 1300° C., for example. If the temperature is less than 280° C., suppression of the leaching may be insufficient, for example the catalyst contains more amorphous titanium oxide. If it is more than 1300° C., sufficient catalyst surface area might not be obtained sufficiently, which fails to produce fatty acid alkyl esters and/or glycerin with high efficiency. More preferably, the lower limit is 400° C., and the upper limit is 1200° C. The calcination time preferably has a lower limit of 30 minutes and an upper limit of 24 hours. More preferably, the lower limit is 1 hour, and the upper limit is 12 hours. Gas phase atmosphere during the calcination is preferably air, nitrogen, argon, oxygen, and hydrogen atmosphere, and also mixed gas thereof. And more preferably, the calcination is carried out under air or nitrogen atmosphere. Particularly if a metal species constituting the ilmenite structure in the metal oxide is oxidized by oxygen in air when calcined in air atmosphere, the metal oxide is preferably calcined in inert gas atmosphere, such as in nitrogen atmosphere in view of stability of the ilmenite crystal structure. If the catalyst, in which the active component consisted of a single or mixed oxide of the metallic element is carried or immobilized on the carrier, is produced, as mentioned above, the catalyst is preferably produced by mixing and carrying the active component on a compound used as a carrier by an impregnation method or a kneading method and the like, and then by calcinating the compound under the above-mentioned calcination conditions. Thus-produced catalyst can disperse the active component sufficiently over the carrier surface to function as a solid catalyst.

Furthermore, the above-mentioned catalyst preferably has insolubility to any of a fat or oil, an alcohol, and a product (fatty acid alkyl esters, glycerin and the like) under reaction conditions (hereinafter referred also to as “insoluble catalyst”). In the reaction of bringing a fat or oil into contact with an alcohol in the presence of the catalyst, the reaction mixture will separates into two phases as the reaction proceeds: one (ester phase) mainly containing a fatty acid alkyl ester; and the other (glycerin phase) mainly containing glycerin, which is a byproduct. In this case, both phases contain the alcohol, and as a result, the fatty acid alkyl ester and the glycerin distribution into two phases. If the alcohol is removed by evaporation in the absence of the catalyst, mutual solubility of the upper layer mainly containing the fatty acid alkyl ester and the lower layer mainly containing the glycerin decreases, which accelerates the separation of the fatty acid alkyl ester from the glycerin. Therefore, recovery ratio can be improved. If the active metal component leaches out from the catalyst, a reverse reaction proceeds in the above-mentioned step and thereby yield of the fatty acid alkyl ester decreases because transesterification is a reversible reaction. As mentioned above, the phase separation after removing the alcohol from the reaction mixture by evaporation in the absence of the catalyst brings about easy purification and high isolated yield during the production method of fatty acid alkyl esters and/or glycerin. That is, preferable embodiment of the present invention includes the production method comprises a step of bringing a fat or oil and an alcohol in the presence of the catalyst, wherein the catalyst is insoluble to any of the fat or oil, the alcohol, and the product (fatty acid alkyl esters, glycerin and the like), and the alcohol is removed by evaporation in the absence of the catalyst before the ester phase and the glycerin phase, which are reaction mixture, are separated. Furthermore, addition of water in minute amounts further improves the separation of the fatty acid alkyl ester from the glycerin and the purification thereof.

The term “the absence of the catalyst” so referred to above means that the reaction product solution hardly contains an insoluble solid catalyst and has a total concentration of active metal components leached from the insoluble solid catalyst of 1000 ppm or less. The term “active metal component leached” means metal components derived from the insoluble solid catalyst eluted into the reaction solution and capable of serving as a homogeneous catalyst with catalytic activity in a transesterification and/or esterification under operation conditions. The leached active metal component having a concentration of more than 1000 ppm fails to suppress the reverse reaction sufficiently in the above-mentioned alcohol-distilled step. Therefore, load of utility in the production can be insufficiently reduced. The concentration is preferably 800 ppm or less, and more preferably 600 ppm or less, and still more preferably 300 ppm or less. Most preferably, the reaction product solution substantially contains no active metal component.

The leaching amount of the above-mentioned active metal components of the catalyst in the reaction solution can be determined by subjecting the reaction solution as it is to fluorescent X-ray spectroscopy (XRF) analysis. Smaller leaching amount is preferably determined by inductively coupled plasma (ICP) emission spectrometry.

The method according to the present invention produces fatty acid alkyl esters and/or glycerin with high efficiency, as mentioned above, because the above-mentioned metal oxide has the following characteristics: performing the esterification reaction and transesterification simultaneously; having no effect from the mineral acid or the metal component contained in the fat or oil; and avoiding decomposition of the alcohol. Such a catalyst used in the production method of the present invention is part of the invention.

The method for producing fatty acid alkyl esters and/or glycerin according to the present invention comprises a step of bringing a fat or oil into contact with an alcohol in the presence of the catalyst. In the contacting step, transesterification reaction of a triglyceride and methanol, for instance, gives a fatty acid methyl ester and glycerin, as shown by the following formula.

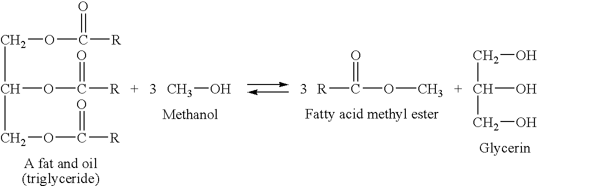

in the formula, R's are the same or different from each other and each represents an alkyl group containing 6 to 24 carbon atoms or an alkenyl group containing 6 to 24 carbon atoms and having one or more unsaturated bonds. More preferably, the alkyl group and the alkenyl group each contain 10 to 22 carbon atoms, and still more preferably contain 12 to 22 carbon atoms.

In the production method, use of the above-mentioned catalyst makes it possible to perform both the transesterification and esterification reactions simultaneously. Therefore, even if a fat or oil, which are raw materials, contain a free fatty acid, yield of a fatty acid alkyl ester can be improved without carrying out the esterification reaction and the transesterification separately, because the esterification reaction of the free fatty acid proceeds simultaneously in the transesterification step.

In the above production method, glycerin is produced in the transesterification reaction together with a fatty acid alkyl ester, as shown by the above formula. In the present invention, purified glycerin can be easily produced as an industrial scale, and such glycerin is useful as a chemical material in various applications.

The fat or oil used in the above-mentioned contacting step contain a fatty acid ester of glycerin and may be any species capable of serving as a raw material for a fatty acid alkyl ester and/or glycerin together with an alcohol. Thus, those generally called “fats and oils” may be used. It is generally preferred that fats and oils containing triglycerides (triesters of higher fatty acids with glycerin) as a main component and containing small amounts of diglycerides, monoglycerides and other minor components. Fatty acid esters of glycerin such as triolein also may be used.

Usable as the above-mentioned fats and oils are vegetable oils such as rapeseed oil, canola oil, sesame oil, soybean oil, corn oil, sunflower oil, palm oil, palm kernel oil, coconut oil, safflower oil, linseed oil, flaxseed oil, cottonseed oil, tung oil, jatropha oil, castor oil, hemp oil, mustard oil, peanut oil and jojoba oil; animal fats and oils such as beef fat, lard, fish oil and whale oil; and various used edible fats and oils (waste cooking oil), among others. One or two or more of these may be used. These fats and oils may contain organic acid, and may be subjected to a pretreatment such as deacidification.

If the above fat or oil contains phospholipids, proteins or the like as impurities, the fat or oil is preferably used after a degumming step of adding a mineral acid such as sulfuric acid, nitric acid, phosphoric acid or boric acid to the fat or oil for removing the impurities therefrom. According to the method for producing fatty acid alkyl esters and/or glycerin of the present invention, performance of the catalyst is hardly inhibited by the mineral acid in the reaction. Therefore, fatty acid alkyl esters and/or glycerin can be produced efficiently, even if the fat or oil after the degumming step contains the mineral acid.

The alcohol used in the above contacting step preferably has 1 to 6 carbon atoms and more preferably 1 to 3 carbon atoms, for production of biodiesel fuel. Employable as the alcohol containing 1 to 6 carbon atoms are, for example, methanol, ethanol, propanol, isopropyl alcohol, 1-butanol, 2-butanol, t-butyl alcohol, 1-pentanol, 3-pentanol, 1-hexanol, 2-hexanol. Among them, methanol is preferred. These may be used singly or in combination of two or more species.

A polyol is also preferable as the above alcohol if the alcohol is used for production of edible oils, cosmetics, medicines and the like.

The above polyol may be preferably ethylene glycol, propylene glycol, glycerin, pentaerythritol, sorbitol or the like. Among them, glycerin is preferred. These may be used singly or in combination of two or more species. Thus, if the polyol is used as the above alcohol, the method for producing fatty acid alkyl esters according to the present invention may be suitably employed for production of glycerides.

In the above method for producing fatty acid alkyl esters and/or glycerin, components other than the fat or oil, the alcohol, and the catalyst may be present.

The alcohol is used in the amount of 1 to 10 times the amount of theoretically needed alcohol in the reaction of the fat or oil and the alcohol. If the amount is less than 1 time, improvement of conversion rate may be insufficient because of insufficient reaction of the fat or oil with the alcohol. If the amount is more than 10 times, excess alcohol to be recovered or to be recycled may be increased, to thereby raise costs. The amount to be used preferably has a lower limit 1.1 times the amount of theoretically needed alcohol in the reaction, more preferably 1.3 times, and most preferably 1.5 times. The amount to be used preferably has an upper limit 9 times the amount of theoretically needed alcohol in the reaction, more preferably 8 times, and most preferably 6 times. The range of the amount to be used is more preferably 1.1 times to 9 times the amount of theoretically needed alcohol in the reaction, more preferably 1.3 times to 8 times, and most preferably 1.5 times to 6 times.

The term “amount of theoretically needed alcohol” means the number of moles of the alcohol corresponding to the saponification value of the source fat or oil, and the amount can be calculated as follows:

[Amount of theoretically needed alcohol (kg)]=[(molecular weight of alcohol)]×[amount to be used of fat or oil (kg)×saponification value (g-KOH/kg-fat or oil)/56100].

If the polyol is used as the above alcohol, diglycerides are produced suitably by the production method of fatty acid alkyl esters according to the present invention. Such an embodiment is part of preferable embodiment of the present invention. The diglycerides produced in such a manner may be suitably used in food fields, for example, as an additive for improving a plasticity of fat or oil. If the diglyceride is converted into an edible fat or oil and then the edible fat or oil is mixed with various foods, it exhibits effects, such as prevention of obesity and inhibition of weight increase. Therefore, use of a diglyceride produced by the present invention as an edible fat or oil is also part of preferable embodiment of the present invention.

In the embodiment for producing diglycerides, a reaction proceeds in the way shown by the following formula if glycerin is used as the polyol, for example.

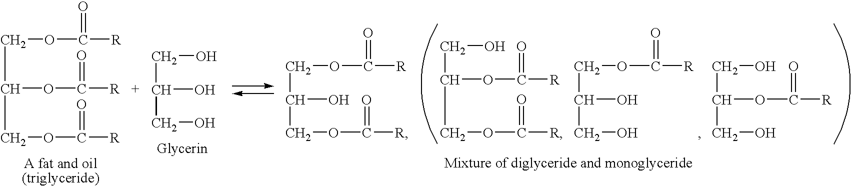

in the formula, R's are the same or different from each other and each represents an alkyl group containing 6 to 22 carbon atoms or an alkenyl group containing 6 to 22 carbon atoms and having one or more unsaturated bonds.

A preferable embodiment of the present invention for producing diglycerides is a method comprising a production of a mixture containing a monoglyceride and a diglyceride as main components in advance, and then carrying out a reaction between the mixture and a free fatty acid or an alkyl ester thereof, which is added to the mixture, in the presence of lipase. Such a method gives diglycerides with high selectivity.

The lipase is preferably immobilized lipase or intracellular lipase. More preferably, the lipase is immobilized lipase or intracellular lipase selectively acting at the 1,3-positions. The immobilized lipase is preferably produced by immobilizing the 1,3-selective lipase onto an ion exchange resin. Preferred examples of the 1,3-selective lipase include lipase derived from microorganism, such as the genus Rhizopus, Aspergillus, Mucor, Rhizomucor, Candida, Thermomyces, or Pseudomonas.

Any condition for allowing the lipase to act on a substrate can be applied as long as good lipase activity is exhibited, but the reaction temperature is preferably 10 to 100° C., and more preferably 20° C. or more and 80° C. or less.

A general method for producing diglycerides, which may be mentioned, is a method comprising a first reaction for reacting a fat or oil with a polyol using a catalyst, and a second reaction for allowing lipase to act on the produced mixture. If an alkali is used as the catalyst in the first reaction, pH of the reaction mixture needs to be adjusted after the completion of the first reaction in order to be within a range suitable for activity of the lipase in the second reaction. However, if the production method of fatty acid alkyl esters according to the present invention is employed as the first reaction, reaction processes can be simplified because need for adjusting pH of the reaction mixture is little in the second reaction. As mentioned above, it is part of preferable embodiment to use the production method of fatty acid alkyl esters according to the present invention as a method for producing diglycerides.

In the method for producing fatty acid alkyl esters and/or glycerin according to the present invention, the reaction temperature has a lower limit of 100° C. and an upper limit of 300° C. If the reaction temperature is less than 100° C., reaction rate might be insufficiently improved. If the reaction temperature is more than 300° C., suppression of a side reaction, such as alcohol decomposition, might be insufficient. More preferably the lower limit is more preferably 120° C. and the upper limit is more preferably 270° C. Still more preferably, the lower limit is 150° C. and the upper limit is 235° C.

In the method for producing fatty acid alkyl esters and/or glycerin, the catalyst preferably contains an active component not leaching in the reaction temperatures within the above range. Use of such a catalyst can maintain the catalyst activity even in high reaction temperatures, and perform the reaction well.

In the method for producing fatty acid alkyl esters and/or glycerin according to the present invention, the reaction pressure preferably has a lower limit of 0.1 MPa and an upper limit of 10 MPa. If the reaction pressure is less than 0.1 MPa, reaction rate might be insufficient. If the reaction pressure is more than 10 MPa, a side reaction might tend to proceed easily. In addition, a special apparatus durable for high pressures may be needed, hence utility costs and equipment costs might not be reduced enough. The lower limit of the reaction pressure is more preferably 0.2 MPa, and still more preferably 0.3 Mpa. The upper limit is more preferably 9 MPa and still more preferably 8 MPa.

As mentioned above, the reaction can be carried out efficiently even if the reaction temperature and pressure are low enough, because of the use of the catalyst with high activity in the method for producing fatty acid alkyl esters and/or glycerin of the present invention.

And the above catalysts (catalysts (I) to (V)) can be used under supercritical condition of an alcohol to be used. The term “supercritical state” means that a region over the specific critical temperature and the critical pressure of a substance. If methanol is used as the alcohol, the state means the temperature of 239° C. or more and the pressure of 8.0 MPa or more. Use of the catalyst permits efficient production of fatty acid alkyl esters and/or glycerin even under the supercritical state.

In the method for producing fatty acid alkyl esters and/or glycerin, the catalyst amount used in the reaction preferably has a lower limit of 0.5% by weight and an upper limit of 20% by weight, relative to the total fed amount of the fat or oil, the alcohol and the catalyst, for example, in a batch method. If the lower limit is less than 0.5% by weight, reaction rate might be insufficiently improved. If the upper limit is more than 20% by weight, catalyst costs might not be reduced enough. The lower limit is more preferably 1.5% by weight, and the upper limit is more preferably 10% by weight. In a fixed bed flow system, a liquid hourly space velocity (LHSV) calculated by the following formula preferably has a lower limit of 0.1 hr⁻¹ and an upper limit of 20 hr⁻¹. More preferably, the lower limit is 0.2 hr⁻¹ and the upper limit is 10 hr⁻¹. Still more preferably, the lower limit is 0.3 hr⁻¹ and the upper limit is 5 hr⁻¹.

LHSV(hr⁻¹)={(flow rate of a fat or oil per hour(L·hr⁻¹))+(flow rate of an alcohol per hour(L·hr⁻¹))}/(volume of a catalyst(L))

The above contacting step is preferably carried out by a batch type (batch-wise) or continuous flow system. Among them, a fixed bed flow system is preferably employed because separation of the catalyst is unnecessary. That is, the contacting step is preferably carried out with a fixed bed and continuous flow reactor. And preferred embodiment of the batch system includes addition of the catalyst into a mixture of the fat or oil and the alcohol.

In the production method of the present invention, the reaction mixture may contain an unreacted raw material, an intermediate glyceride and the like because use of the catalyst enables the reaction to be repeated. In this case, it is preferable to recover the unreacted glyceride and the free fatty acid, and to reuse the recovered glyceride and the free fatty acid with the fat or oil. The recovery of such unreacted glyceride and free fatty acid can be carried out, for example, by removing low-boiling components, such as alcohol and water from the mixture after the reaction in the absence of the catalyst, and then separating the unreacted glyceride and the free fatty acids from the effluent. This procedure makes it possible to produce a high-purity fatty acid alkyl ester and glycerin with high yield and to reduce purification costs more efficiently.

The fatty acid alkyl esters produced by the method according to the present invention are used suitably for various uses as, for example, an industrial raw material, a raw material for pharmaceuticals, fuels and the like. Among them, diesel fuel containing fatty acid alkyl esters made from vegetable fats and oils, or waste cooking oil produced by the above production method can sufficiently reduce utility costs and equipment costs in the production step. Furthermore, no need for recovery step of the catalyst permits a repeating use of the catalyst. Therefore, diesel fuel can sufficiently contribute to environmental preservation in the production step, and suitably used for various applications. Such diesel fuel containing the fatty acid alkyl ester produced by the above production method is also part of the present invention.

FIGS. 1 and 2 each show a preferable embodiment of the production steps in the method for producing fatty acid alkyl esters and/or glycerin according to the present invention. However, the present invention is not limited to these embodiments. FIG. 1 shows a step of bringing palm oil as the fat or oil into contact with methanol as the alcohol by a batch method in the presence of a catalyst. In such an embodiment, the palm oil and the methanol are mixed with the catalyst to be subjected to reaction. This reaction mixture is kept standing and separated into an ester phase mainly containing a fatty acid methyl ester and a glyceride and a glycerin phase mainly containing glycerin and methanol. Then, methanol and the catalyst are added to the ester phase separated from the glycerin phase to cause further reaction, and the obtained mixture is separated into an ester phase and a glycerin phase. Then, a fatty acid methyl ester and glycerin are finally produced. In such an embodiment, the alcohol is preferably removed before separating the reaction mixture into two phases and after separating and removing the solid catalyst from the liquid phase by, for example, a filtration step, because separation of the fatty acid methyl ester and the glycerin can be improved. It is preferable that thus-produced fatty acid methyl ester and/or glycerin are further purified by distillation and the like depending on purpose.

FIG. 2 shows a step of bringing palm oil as the fat or oil, and methanol as the alcohol into contact with a solid catalyst, by using continuous-flow fixed-bed reactor. The mixture reacted in the reaction column filled with the catalyst is kept standing in a settler to separate into an ester phase and a glycerin phase. The ester phase separated from the glycerin phase is further reacted with methanol in the reaction column filled with the catalyst to produce the reaction mixture. Then, methanol is removing from the reaction mixture by evaporation, thus obtained mixture is kept standing in the settler to separate into an ester phase and a glycerin phase. Thereby, a fatty acid methyl ester and glycerin are finally produced. It is preferable that thus-produced fatty acid methyl ester and/or glycerin are further purified by distillation and the like depending on purpose.

The present invention also relates to a catalyst for producing fatty acid alkyl esters and/or glycerin, wherein the catalyst is used in the method for producing fatty acid alkyl esters and/or glycerin. Such a catalyst is preferably at least one of the following catalysts (I) to (V):

(I) a metal oxide having an ilmenite structure and/or a slyrankite structure;

(II) an oxide containing a metallic element belonging to the Group 12 and a metallic element belonging to the Group 4;

(III) a mixed oxide containing a metallic element belonging to the Group 12 and a tetravalent transition metallic element;

(IV) a metal oxide containing zirconium and at least one metallic element selected from the group consisting of metallic elements belonging to the Groups 4, 5 and 8; and

(V) a metal oxide containing anatase type titanium oxide and/or rutile type titanium oxide, and the metal oxide containing a sulfur component of 700 ppm or less.

Among them, the catalyst is more preferably a metal oxide having an ilmenite structure, or an oxide having a cubic structure and having metal elements belonging to the Groups 12 and 4. As a configuration, structure, production method, concrete example and the like of the above catalyst, those mentioned above is preferred. Most preferred are MnTiO₃, CoTiO₃, ZnTiO₃, FeTiO₃, and NiTiO₃ each having an ilmenite structure; cubic ZnZrO₃; and Zn₂TiO₄ having a spinel structure. If such a catalyst is used in the above-mentioned production method, no leaching occurs even if the catalyst is repeatedly used in the reaction, and the catalyst can be used for a long term. And use of the catalyst can make the separation and removal step of the catalyst simple, which is economically excellent.

The method for producing fatty acid alkyl esters and/or glycerin according to the present invention has the above-mentioned configurations. Therefore, the method has the following advantages.

In view of simplification of the reaction processes,

(1) The separation and removal step of the catalyst can be simplified or omitted;
(2) The neutralization and removal step of the free fatty acids, or the esterification step with the acid catalysts, is eliminated;
(3) No saponification of the free fatty acids occurs; and
(4) Esterification of the free fatty acids in the fat or oil proceeds simultaneously.

In view of simplification of the purification processes, that is, easier production of purified glycerin,

(1) The alcohol can be evaporated after separation of the catalyst, and the distribution equilibrium of liquid-liquid two phases can be improved (mutual solubility decreases) because of no reverse reaction, and therefore, the product can be separated well; and
(2) No leaching of the catalyst occurs and the catalyst life can be extended, because the catalyst (at least one of the above-mentioned catalysts (I) to (V)) uses the essential metal component in the crystal lattice as an activate species; the fatty acid alkyl ester can be produced with high selectivity, because the degradation of the alcohol and/or glycerin by dehydration or coking can be negligible due to absence of strong acid-site or base-site on the catalyst surface; and the catalyst is hardly affected by a small amount of the metal components or the mineral acids used for the pretreatment.

### BRIEF DESCRIPTION OF THE DRAWINGS

FIG. 1 is a view schematically showing a preferable embodiment of the production steps in the method for producing fatty acid alkyl esters and/or glycerin according to the present invention.

FIG. 2 is a view schematically showing a preferable embodiment of the production steps in the method for producing fatty acid alkyl esters and/or glycerin according to the present invention.

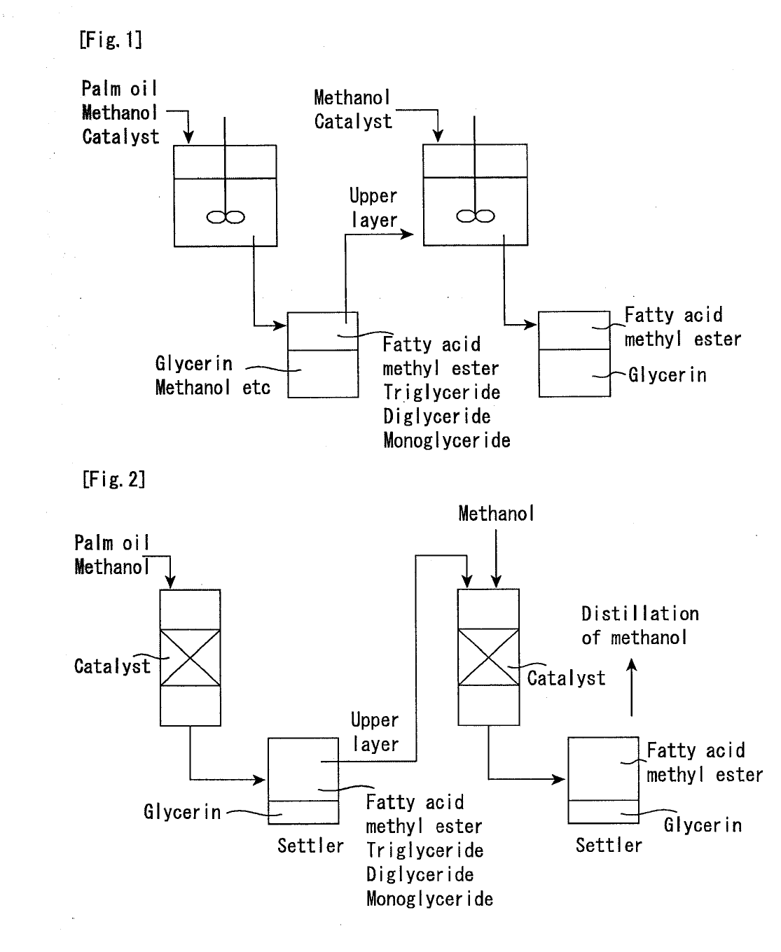

### BEST MODE FOR CARRYING OUT THE INVENTION

The present invention will, hereinafter, be described in more detail with reference to Examples, but the present invention is not limited to these Examples.

Conversion rates and yields were calculated according to the following formulae.

(Conversion rate(%))=(number of moles of consumed fat or oil at the completion of a reaction)/(number of moles of fed fat or oil)×100(%)

(Yield of methyl ester(% by mole))={(number of moles of produced methyl ester at the completion of a reaction)/(number of moles of fed effective fatty acids)}×100(%)

(Yield of diglycerides(% by mole))={[(number of moles of produced diglycerides at the completion of a reaction)×2]/(number of moles of fed effective fatty acids)}×100(%)

(Yield of monoglycerides(% by mole))={(number of moles of monoglycerides at the completion of a reaction)/(number of moles of fed effective fatty acids)}×100(%)

(Yield of glycerin(% by mole))={(number of moles of produced isolation glycerin at the completion of a reaction)/(number of moles of fed effective glycerin components)}×100(%)

The term “effective fatty acids” means triglycerides, diglycerides, monoglycerides, and free fatty acids of fatty acids contained in the fat or oil. That is, the number of moles of fed effective fatty acids is calculated by the following formula.

(Number of moles of fed effective fatty acids)=[(amount of fed fat or oil(g))×(saponification value of fat or oil(mg-KOH/g-fat or oil))/56100]

The term “effective glycerin components” means those components capable of producing glycerin by the method of the present invention, and specifically, triglycerides diglycerides, and monoglycerides of fatty acids contained in the fat or oil. The content of the effective glycerin components can be determined by gas chromatography of free glycerin produced by saponification of the fat or oil (reactant).

### CATALYST PREPARATION EXAMPLE 1

### Preparation of MnTiO₃ Catalyst

Manganese carbonate (MnCO₃) 200 g and anatase type titanium oxide (TiO₂) 150 g were powder-mixed, and the mixture was calcined at 1000° C. for 5 hours under air flow to give MnTiO₃ catalyst 273 g. XRD analysis of the catalyst showed that the catalyst had an ilmenite structure and contained small amount of rutile type TiO₂.

### CATALYST PREPARATION EXAMPLE 2

### Preparation of ZnTiO₃ (Ilmenite Type) Catalyst

Zinc oxide (ZnO) 20 g and anatase type titanium oxide (TiO₂) 20 g were powder-mixed. The mixture was calcined at 700° C. for 4 hours under air flow to give ZnTiO₃ catalyst. XRD analysis of the catalyst showed that the catalyst was ZnTiO₃ having an ilmenite structure.

### CATALYST PREPARATION EXAMPLE 3

### Preparation of ZnZrO₃ Catalyst

Zinc nitrate (Zn(NO₃)₂) 45 g and a 25% aqueous solution of zirconium oxynitrate (ZrO(NO₃)₂) 74 g were mixed, and pure water was added until the total amount became 300 mL. To this aqueous solution was added 180 mL of an aqueous solution of 1 mol/L oxalic acid, and then the mixture was stirred at 70° C. for 3 days. The precipitation generated was recovered by centrifugal separation, and then the recovered precipitation was calcined at 600° C. for 5 hours to give zinc zirconate catalyst. XRD analysis of the catalyst showed that the catalyst contained cubic ZnZrO₃.

### CATALYST PREPARATION EXAMPLE 4

### Preparation of Zn₂TiO₄ Catalyst

Zinc oxide (ZnO) 20 g and anatase type titanium oxide (TiO₂) 10 g were powder-mixed. The mixture was calcined at 1000° C. for 4 hours under air flow to give Zn₂TiO₄ catalyst. XRD analysis of the catalyst showed that the catalyst was Zn₂TiO₄ having a spinel structure.

### CATALYST PREPARATION EXAMPLE 5

### Preparation of Fe—V—Zr Catalyst

Oxalic acid dihydrate 6.49 g was dissolved in water 46.84 g, and thereto vanadium pentaoxide 3.12 g was added and uniformly dissolved therein (solution A). Iron (III) nitrate nonahydrate 13.86 g and zirconyl nitrate (IV) dihydrate 74.08 g were dissolved in water 740 g, and thereto the solution A was added. Then the mixture was stirred for 2 hours at room temperature. After evaporation to dryness, the residue was preliminarily calcined at 350° C. under air flow for 2 hours and then calcined at 750° C. for 5 hours to give FeVZr catalyst. Contents of iron, vanadium, and zirconium were respectively 4.8% by weight, 4.4% by weight, and 63.1% by weight in metal conversion, relative to 100% by weight of the catalyst. XRD analysis of the catalyst showed that the catalyst was principally a mixture containing a composite oxide with a triclinic FeVO₄ structure and monoclinic zirconium oxide.

### CATALYST PREPARATION EXAMPLE 6

### Preparation of Ti—Zr Catalyst

Zirconium oxide 60 g (RSC-HP, product of DAIICHI KIGENSO KAGAKU KOGYO Co., LTD.) was kneaded in a solution prepared by homogeneously dissolving titanium tetraisopropoxide 17 g in isopropyl alcohol 2.3 g, and the mixture was then evaporated to dryness with stirring. The residue was dried at 120° C. for overnight, and then calcined at 900° C. for 3 hours under air flow to give Ti—Zr catalyst. The catalyst was subjected to X-ray fluorescence analysis by a glass bead method to find that sulfur component was 50 ppm or less, which was blow detection limit and that contents of titanium and zirconium were respectively 4.4% by weight and 68.6% by weight in metal conversion, relative to 100% by weight of the total amount of the catalyst. XRD analysis of the catalyst showed that the catalyst contained rutile type titanium oxide and monoclinic zirconium oxide.

### CATALYST PREPARATION EXAMPLE 7

### Preparation of Ti—Si Catalyst

### Synthetic Example Using Titanium Tetraisopropoxide

Silica powder 25 g (CARiACT Q-50, product of Fuji Silysia Chemical Ltd., particle diameter of 75 to 180 μm) was kneaded in a solution prepared by diluting titanium tetraisopropoxide 7.1 g with isopropyl alcohol 11.5 g, and the mixture was then evaporated to dryness with stirring. The residue was calcined at 600° C. for 4 hours under air flow to give Ti—Si catalyst. The catalyst was subjected to X-ray fluorescence analysis by a glass bead method to find that sulfur component was 50 ppm or less, which was below detection limit, and that contents of titanium and silicon were respectively 4.4% by weight and 43.3% by weight in metal conversion, relative to 100% by weight of the total amount of the catalyst. XRD analysis of the catalyst showed that the titanium oxide contained in the catalyst was anatase type.

### CATALYST PREPARATION EXAMPLE 8

### Preparation of Ti—Si Catalyst

### Synthetic Example Using Titanium Oxysulfate

Silica powder 80 g (CARiACT Q-50, product of Fuji silysia Chemical Ltd., particle diameter of 75 to 180 μm) was kneaded in a solution prepared by dissolving titanium oxysulfate 14.5 g (Ti content: 33.21% by weight as TiO₂) in water 140 g, and the mixture was then evaporated to dryness with stirring. The residue was calcined at 600° C. for 4 hours under air flow and sufficiently washed with water and dried to give Ti—Si catalyst. The catalyst was subjected to X-ray fluorescence analysis by a glass bead method to find that sulfur component was 50 ppm or less, which was below detection limit, and contents of titanium and silicon were respectively 3.4% by weight and 44.1% by weight in metal conversion, relative to 100% by weight of the total amount of the catalyst. XRD analysis of the catalyst showed that the titanium oxide contained in the catalyst was anatase type.

### CATALYST PREPARATION EXAMPLE 9

### Preparation of Ti—Al Catalyst

α-alumina 30 g (SA5151, product of SAINT-GOBAIN) was impregnated with a solution prepared by diluting titanium tetraisopropoxide 2.4 g with isopropyl alcohol 5.0 g, and the mixture was then evaporated to dryness with stirring. The residue was dried at 120° C. for overnight, and then further calcined at 900° C. for 3 hours under airflow to give Ti—Al catalyst. The catalyst was subjected to X-ray fluorescence analysis by a glass bead method to find that sulfur components was 50 ppm or less, which was below detection limit, and contents of titanium and aluminum were respectively 4.4% by weight and 51.8% by weight in metal conversion, relative to 100% by weight of the total amount of the catalyst. XRD analysis of the catalyst showed that the titanium oxide contained in the catalyst was rutile type.

### CATALYST FOR COMPARISON PREPARATION EXAMPLE 1

### Preparation of ZnAl₂O₄ Catalyst

Zinc oxide 63.8 g and alumina gel containing alumina 136.2 g were mixed in an aqueous solution of nitric acid, and the mixture was calcined at 400° C. for 2 hours. XRD analysis of the residue showed that the residue contained ZnAl₂O₄ in the crystal structure.

### CATALYST FOR COMPARISON PREPARATION EXAMPLE 2

### Preparation of TiVO₄ Catalyst

A 20% aqueous solution of titanium trichloride (III) 169.66 g was added dropwise into a solution prepared by dissolving ammonium metavanadate 25.74 g into distilled water 700 g at 90° C. After evaporation to dryness, the residue was preliminarily calcined at 350° C. under air flow for 2 hours and then calcined at 750° C. for 5 hours. XRD analysis of the residue showed that the residue contained TiVO₄ having a rutile structure.

### CATALYST FOR COMPARISON PREPARATION EXAMPLE 3

### Preparation of FeVO₄ Catalyst

Ammonium metavanadate 14.04 g was dissolved in water 700 g at 90° C. (solution B). A solution prepared by dissolving iron (III) nitrate nonahydrate 48.48 g in water 40 g was added dropwise into the solution B, and the mixture was then evaporated to dryness. The residue was preliminarily calcined, under air flow, at 350° C. for 2 hours and then calcined at 750° C. for 5 hours to give FeVO₄ catalyst. XRD analysis of the catalyst showed that the catalyst was principally a composite oxide with a triclinic FeVO₄ structure.

### CATALYST FOR COMPARISON PREPARATION EXAMPLE 4

### Preparation of K/ZrO₂ Catalyst

Potassium carbonate 20 g and zirconium oxide 80 g (product of DAIICHI KIGENSO KAGAKU KOGYO Co., Ltd.) were powder-mixed, and a small amount of water was added, and the mixture was further kneaded thoroughly. After drying, the residue was calcined at 550° C. for 4 hours under air flow to give K/ZrO₂ catalyst.

### CATALYST FOR COMPARISON PREPARATION EXAMPLE 5

### Preparation of Ti—Si Catalyst (Synthetic Example Using Titanium Oxysulfate, without Rinsing)

Silica powder 60 g (CARiACT Q-50, product of Fuji silysia Chemical Ltd., particle diameter of 75 to 180 μm) was kneaded in a solution prepared by dissolving titanium oxysulfate 14.5 g (Ti content: 33.21% by weight as TiO₂) in water 140 g, and the mixture was then evaporated to dryness with stirring. The residue was calcined at 600° C. for 4 hours under air flow to give Ti—Si catalyst. The catalyst was subjected to X-ray fluorescence analysis by a glass bead method to detect 950 ppm of sulfur component.

### EXAMPLE 1

A reaction of palm oil with methanol was carried out using a fixed bed and continuous flow reactor. A straight reactor tube made of stainless steel (SUS316) having 10 mm in inside diameter and 210 mm in length was charged with 15 mL (12 g) of MnTiO₃ catalyst obtained from Catalyst Preparation Example 1. Palm oil and methanol were continuously flowed into the reactor tube at flow rates of 0.12 mL/min and 0.13 mL/min (LHSV-1 hr⁻¹, methanol: 9 times the amount of theoretically needed) respectively, using a precision and high-pressure metering pump. The reactor tube was installed in a heating oven to set a temperature inside the oven at 200° C. And the reactor tube outlet was equipped with a back pressure regulator via an air-cooled condenser, and the pressure within the reactor tube was set at 5 MPa. Yields of fatty acid methyl ester and glycerin were respectively 91.9 mol % and 85.0 mol % in the reactor tube outlet 10 hours after stable operation of the fixed bed and continuous flow reactor. Conversion rate of triglyceride, which was main component of the fat or oil, was 96.7 mol %, and yields of diglyceride and monoglyceride were respectively 2.8 mol % and 8.9 mol %. The reaction mixture in the outlet was subjected to the ICP emission spectrometry to detect neither Mn nor Ti, which were catalyst components. This reaction was continuously repeated for 250 hours, and the reaction mixture in the outlet was analyzed. No change was observed in composition of the reaction mixture in the outlet, which indicates the catalyst does not deteriorate with age and therefore it has a long life.

### EXAMPLE 2

A 200-mL autoclave was charged with palm oil 40 g, methanol 40 g, and MnTiO₃ catalyst powder 3 g prepared in Catalyst Preparation Example 1. After nitrogen replacement, the reaction was allowed to proceed at a reaction temperature of 200° C. for 24 hours with internal stirring. The inside was cooled up to 50° C. to take out the reaction mixture. The reaction mixture was subjected to centrifugal separation to separate the catalyst. Methanol was distilled off with a rotating evaporator. Then, the resultant was separated into methyl ester phase (upper phase) and glycerin phase (lower phase) to recover the upper phase. A 200-mL autoclave was charged with methanol in the same amount of the recovered upper layer and MnTiO₃ catalyst powder 3 g prepared in Catalyst Preparation Example 1. And the reaction was again allowed to proceed at a reaction temperature of 200° C. for 24 hours in the same manner as in the first reaction. Yields of fatty acid methyl ester and glycerin were 99.6 mol % and 98.7 mol %, respectively. Triglyceride and diglyceride, which were main components of the fat or oil, were completely converted. Yield of monoglyceride was 1.1 mol %. The reaction mixture was subjected to the ICP emission spectrometry to detect neither Mn nor Ti, which were catalyst components.

### EXAMPLE 3

A 200 mL autoclave was charged with triolein 60 g, methanol 20 g and MnTiO₃ catalyst (product of Alfa Aesar Co.) 2.5 g. XRD analysis of the MnTiO₃ catalyst used in this Example showed that the catalyst had an ilmenite structure. After nitrogen replacement, the reaction was allowed to proceed at a reaction temperature of 200° C. for 24 hours with internal stirring. Conversion rate of triolein was 99%, and yields of methyl oleate, monoglyceride, diglyceride, and glycerin were respectively 87%, 10%, 2%, and 49%. Neither by production of glycerin condensates or etherification products nor decomposition of methanol was observed. ICP emission spectrometry of the reaction mixture showed that the concentration of leached Ti was 1 ppm or less, and that of leached Mn was 20 ppm or less.

### EXAMPLE 4

A reaction was carried out in the same manner as in Example 3, except that CoTiO₃ catalyst (product of Alfa Aesar Co.) was used as a catalyst. XRD analysis of the CoTiO₃ catalyst used in this Example showed that the catalyst had an ilmenite structure. Conversion rate of triolein was 96%, and yields of methyl oleate, monoglyceride, diglyceride, and glycerin were respectively 77%, 17%, 2%, and 41%. ICP emission spectrometry of the reaction mixture showed that the concentrations of leached Ti and Co were each 1 ppm or less.

### EXAMPLE 5

A reaction was carried out in the same manner as in Example 3, except that FeTiO₃ catalyst (product of Aldrich Co.) was used as a catalyst. XRD analysis of FeTiO₃ catalyst used in this Example showed that the catalyst had an ilmenite structure. Conversion rate of triolein was 98%, and yields of methyl oleate, monoglyceride, diglyceride, and glycerin were respectively 94%, 4%, 0%, and 94%. ICP emission spectrometry of the reaction mixture showed that the concentrations of leached Ti and Fe were each 1 ppm or less.

### EXAMPLE 6

A reaction was carried out in the same manner as in Example 3, except that NiTiO₃ catalyst (product of City Chemical LLC) was used as a catalyst. XRD analysis of the NiTiO₃ catalyst used in this Example showed that the catalyst had an ilmenite crystal structure. Conversion rate of triolein was 97%, and yields of methyl oleate, monoglyceride, diglyceride, and glycerin were respectively 63%, 29%, 5% and 63%. ICP emission spectrometry of the reaction mixture showed that the concentrations of leached Ti and Ni were each 1 ppm or less.

### EXAMPLE 7

A 200-mL autoclave was charged with triolein 61.5 g, methanol 20 g, and ZnTiO₃ catalyst 2.5 g prepared in Catalyst Preparation Example 2. After nitrogen replacement, the reaction was allowed to proceed at a reaction temperature of 200° C. for 24 hours with internal stirring. Conversion rate of triolein was 96%, and yields of methyl oleate and glycerin were respectively 82% and 37%. No leaching of the metals in the reaction mixture was detected by ICP emission spectrometry.

### EXAMPLE 8

Reactions were carried out three times in the same manner as in Example 7, and the same catalyst as in Example 7 was repeatedly used for the reactions. After a second reaction, conversion rate of triolein was 94%, and yields of methyl oleate and glycerin were respectively 77% and 37%. After a third reaction, conversion rate of triolein was 97%, and yields of methyl oleate and glycerin were respectively 84% and 37%. No change was observed in the activity of the catalyst after the repeated use, which indicates that the catalyst has high stability. No leaching of the metals in the reaction mixture was detected after each reaction.

### EXAMPLE 9

A 100-mL autoclave was charged with triolein 30 g, methanol 10 g and a catalyst having a slyrankite structure (Zr_{0.5}Ti_{0.5}O₂, product of Alfa Aesar Co.) 1.25 g. XRD analysis of the Zr_{0.5}Ti_{0.5}O₂ catalyst used in this Example showed that the catalyst had a slyrankite structure. After nitrogen replacement, the reaction was allowed to proceed at a reaction temperature of 200° C. for 24 hours with internal stirring. Conversion rate of triolein was 95%, and yields of methyloleate, monoglyceride, diglyceride, and glycerin were respectively 69%, 18%, 8%, and 63%. ICP emission spectrometry of the reaction mixture showed that leached metal components contained no Zr, and 1 ppm or less of Ti.

### COMPARATIVE EXAMPLE 1

A reaction was carried out in the same manner as in Example 3, except that hydrotalcite was used as a catalyst and the reaction temperature was 150° C. Conversion rate of triolein was 99%, and yields of methyl oleate, monoglyceride, diglyceride, and glycerin were respectively 95%, 2%, 2%, and 89%. XRF analysis of the reaction mixture showed that almost total amount of magnesium constituting the hydrotalcite and about half amount of the aluminum were leached.

### EXAMPLE 10

A reaction was carried out in the same manner as in Example 7, except that ZnZrO₃ catalyst 2.5 g prepared in Catalyst Preparation Example 3 was used as a catalyst. Conversion rate of triolein was 99%, and yields of methyl oleate and glycerin were respectively 84% and 49%. No leaching of the metals in the reaction mixture was detected by ICP emission spectrometry.

### EXAMPLE 11

A reaction was carried out in the same manner as in Example 10, except that Zn₂TiO₄ catalyst 2.5 g prepared in Catalyst Preparation Example 4 was used as a catalyst. Conversion rate of triolein was 97%, and yields of methyl oleate and glycerin were respectively 82% and 43%. No leaching of the metals in the reaction mixture was detected by ICP analysis.

### EXAMPLE 12

Reactions were carried out three times in the same manner as in Example 11, and the same catalyst as in Example 11 was repeatedly used for the reactions. After a second reaction, conversion rate of triolein was 97%, and yields of methyl oleate and glycerin were respectively 84% and 46%. After a third reaction, conversion rate of triolein was 97%, and yields of methyl oleate and glycerin were respectively 84% and 49%. No. change was observed in the activity of the catalyst after the repeated use, which indicates that the catalyst has high stability. No leaching of the metals in the reaction mixture was detected after each reaction.

### COMPARATIVE EXAMPLE 2

A reaction was carried out in the same manner as in Example 8, except that ZnAl₂O₄ catalyst prepared in Catalyst for comparison Preparation Example 1 was used as a catalyst. Conversion rate of triolein was 97%, and yields of methyl oleate and glycerin were respectively 76% and 42%. Infrared (IR) spectral data showed the presence of soap components, and the active component leached.

### COMPARATIVE EXAMPLE 3

Reactions were carried out three times in the same manner as in Example 8, except that TiVO₄ catalyst prepared in Catalyst for comparison Preparation Example 2 was used as a catalyst, the reaction time was 1 hour, and the reaction temperature was 150° C. And the catalyst was repeatedly used for the reactions. After a first reaction, conversion rate of triolein was 97%, and yields of methyl oleate and glycerin were respectively 70% and 29%. After a second reaction, conversion rate of triolein was 94%, and yields of methyl oleate and glycerin were respectively 59% and 19%. After a third reaction, conversion rate of triolein was 89%, and yields of methyl oleate and glycerin were respectively 47% and 10%. The activity of the catalyst decreased after the repeated reactions, which indicates that the catalyst deteriorates. No leaching was detected in the reaction mixture.

### EXAMPLE 13

A 200-mL autoclave was charged with triolein 60 g, methanol 20 g, and Fe—V—Zr catalyst 2.5 g prepared in Catalyst Preparation Example 5. After nitrogen replacement, the reaction was allowed to proceed at a reaction temperature of 150° C. for 24 hours with internal stirring. Yields of methyl oleate and glycerin were respectively 54% and 11%. Neither by production of glycerin condensates or etherification products nor decomposition of methanol was observed. The reaction mixture was measured for metal leaching by ICP emission spectrometry to find that content of leached metal was below detection limit.

### EXAMPLE 14

A 200-mL autoclave was charged with triolein 61.5 g, methanol 20 g, and Fe—V—Zr catalyst 2.5 g prepared in Catalyst Preparation Example 5. After nitrogen replacement, reaction was allowed to proceed at a reaction temperature of 150° C. for 3 hours with internal stirring. Conversion rate of triolein was 78%, and yields of methyl oleate and glycerin were respectively 35% and 3%.

After the reaction, the catalyst was recovered and the recovered catalyst was used again for a reaction under the same conditions. Conversion rate of triolein was 68%, and yields of methyl oleate and glycerin were respectively were 29% and 2%. The catalyst was recovered again after the reaction, and the recovered catalyst was used for a reaction under the same conditions. Conversion rate of triolein was 70%, and yields of methyl oleate and glycerin were 30% and 5%. The activity of the catalyst after three times reactions was not remarkably reduced.

### COMPARATIVE EXAMPLE 4

A reaction was carried out in the same manner as in Example 14, except that FeVO₄ catalyst 2.5 g prepared in Catalyst for comparison Preparation Example 3 was used as a catalyst. Conversion rate of triolein was 83%, and yields of methyl oleate and glycerin were respectively 31% and 5%. After the reaction, the catalyst was recovered and the recovered catalyst was used again for a reaction under the same conditions. Conversion rate of triolein was 35%, and yields of methyl oleate and glycerin were respectively 10% and 0%. The activity of the catalyst after two reactions was reduced.

### EXAMPLE 15

A 100-mL autoclave was charged with triolein 30 g, methanol 10 g and Ti—Zr catalyst 1.25 g prepared in Catalyst Preparation Example 6. After nitrogen replacement, the reaction was allowed to proceed at a reaction temperature of 200° C. for 24 hours with internal stirring. Conversion rate of triolein was 99%, and yields of methyl oleate, monoglyceride, diglyceride, and glycerin were respectively 79%, 17%, 3%, and 79%. Neither by production of glycerin condensates or etherification products nor decomposition of methanol was observed. ICP emission spectrometry of the reaction mixture showed that leached metal components contained no Zr, and 1 ppm or less of Ti.

### EXAMPLE 16

A straight reactor tube made of SUS316 stainless steel having 10 mm in inside diameter and 210 mm in length was charged with 15 mL of Ti—Zr catalyst prepared in Catalyst Preparation Example 6 after pulverized and then classified into 300 to 850 μm. In order to control pressure, a filter and a back pressure regulator were fixed in the reactor outlet with an air-cooled cooling tube therebetween.

Pressure inside the reactor tube was set at 5.0 MPa with the back pressure regulator, while circulating triolein and methanol to a reactor at flow rates of 0.127 mL/min and 0.048 mL/min (molar ratio=1/9, LHSV=0.70 hr⁻¹) respectively, using a precision and high-pressure metering pump. The reactor tube was heated from the outside using GC (gas chromatography) oven to set a temperature at 200° C. Yields of methyl oleate and glycerin were respectively 61% and 38% in the reaction outlet 17 hours after stabilization of the temperature and the pressure. Yields of methyl oleate and glycerin more 50 hours later were respectively 62% and 40%. After that, no decrease of the catalyst activity was observed over 160 hours or more.

### COMPARATIVE EXAMPLE 5

A reaction was carried out in the same manner as in Example 15, except that K/ZrO₂ catalyst prepared in Catalyst for comparison Preparation Example 4 was used as a catalyst, and the reaction temperature was 150° C. Conversion rate of triolein was 99%, yields of methyl oleate, monoglyceride, diglyceride, and glycerin were respectively 95%, 2%, 1%, 90%. XRF analysis of the potassium components leached in the reaction mixture showed that almost total amount of the potassium components were leached. Therefore, it was impossible to use the catalyst repeatedly.

### EXAMPLE 17

A reaction was carried out in the same manner as in Example 16, except that Ti—Si catalyst prepared in Catalyst Preparation Example 7 was used as a catalyst, two reactor tubes each charged with 15 mL of the catalyst were directly connected to set the total volume of the catalyst at 30 mL, the flow rates of triolein and methanol were respectively 0.206 mL/min and 0.078 mL/min (molar ratio=1/9, LHSV=0.57 h r⁻¹), and the reaction pressure was 2.5 MPa. Yields of methyl oleate and glycerin were respectively 54% and 21% in the reaction outlet 17 hours after stabilization of the temperature and the pressure. Yields of methyl oleate and glycerin more 50 hours later were respectively 53% and 18%. After that, no decrease of the catalyst activity was observed over 160 hours or more.

### EXAMPLE 18

A 200-mL autoclave was charged with triolein 60 g, methanol 20 g and Ti—Si catalyst 2.5 g prepared in Catalyst Preparation Example 8. After nitrogen replacement, the reaction was allowed to proceed at a reaction temperature of 200° C. for 24 hours with internal stirring. Yields of methyl oleate and glycerin were respectively 74% and 42%.

### COMPARATIVE EXAMPLE 6

A reaction was carried out in the same manner as in Example 17, except that Ti—Si catalyst prepared in Catalyst for comparison Preparation Example 5 was used as a catalyst. Yields of methyl oleate and glycerin were respectively 14% and 3% in the reaction outlet 17 hours after stabilization of the temperature and the pressure. Yields of methyl oleate and glycerin more 50 hours later were respectively 12% and 2%, which indicates that the catalyst has an extremely low activity.

### EXAMPLE 19

A reaction was carried out in the same manner as in Example 18, except that Ti—Al catalyst prepared in Catalyst Preparation Example 9 was used as a catalyst. Yields of methyl oleate and glycerin were respectively 70% and 40%.

### INDUSTRIAL APPLICABILITY

The method for producing fatty acid alkyl esters and/or glycerin according to the present invention has the above-mentioned configurations. Therefore, the method has the following advantages.

In view of simplification of the reaction processes,

(1) The separation and removal step of the catalyst can be simplified or omitted;
(2) The neutralization and removal step of the free fatty acids, or the esterification step with the acid catalysts, is eliminated;
(3) No saponification of the free fatty acids occurs; and
(4) Esterification of the free fatty acids in the fat or oil proceeds simultaneously.

In view of simplification of the purification processes, that is, easier production of purified glycerin,

(1) The alcohol can be distilled after separation of the catalyst, and the partition equilibrium of liquid-liquid two phases can be improved (mutual solubility decreases) because of no reverse reaction, and therefore, the product can be separated well; and
(2) No leaching of the catalyst occurs and the catalyst life can be extended, because the catalyst (at least one of the above-mentioned catalysts (I) to (V)) uses the essential metal component in the crystal skeleton as an activate species; the fatty acid alkyl ester can be produced with high selectivity, because the alcohol is hardly decompounded (dehydrated or caulked) due to absence of strong acid point or base point on the catalyst surface; and the catalyst is hardly affected by a small amount of the metal components or the mineral acids used for the pretreatment.

This present application claims priority on Patent Application No. 2005-043619, “METHOD FOR PRODUCING GLYCERIN AND/OR FATTY ACID ALKYL ESTERS” filed in Japan on Feb. 21, 2005, Patent Application No. 2005-043620, “METHOD FOR PRODUCING FATTY ACID ALKYL ESTERS AND/OR GLYCERIN” filed in Japan on Feb. 21, 2005, Patent Application No. 2005-043621, “METHOD FOR PRODUCING GLYCERIN AND/OR FATTY ACID ALKYL ESTERS” filed in Japan on Feb. 21, 2005, and Patent Application No. 2005-368602, “METHOD FOR PRODUCING FATTY ACID ALKYLESTERS AND/OR GLYCERIN USING A FAT AND OIL” filed in Japan on Dec. 21, 2005, the entire contents of which are hereby incorporated by reference.

1. A method for producing fatty acid alkyl esters and/or glycerin comprising a step of bringing a fat or oil into contact with an alcohol in the presence of a catalyst,
wherein the catalyst is at least one catalyst selected from the group consisting of the following (I) to (V):
(I) a metal oxide having an ilmenite structure and/or a slyrankite structure;
(II) an oxide containing a metallic element belonging to the Group 12 and a metallic element belonging to the Group 4;
(III) a mixed oxide containing a metallic element belonging to the Group 12 and a tetravalent transition metallic element;
(IV) a metal oxide containing zirconium and at least one metallic element selected from the group consisting of metallic elements belonging to the Groups 4, 5 and 8; and
(V) a metal oxide containing anatase type titanium oxide and/or rutile type titanium oxide, and the metal oxide containing a sulfur component of 700 ppm or less.

2. The method for producing fatty acid alkyl esters and/or glycerin according to claim 1,
wherein the catalyst contains a metal oxide having an ilmenite structure containing titanium and at least one metallic element selected from the group consisting of metallic elements belonging to the Groups 7, 8, 9, 10, and 12.

3. The method for producing fatty acid alkyl esters and/or glycerin according to claim 1,
wherein the oxide is a mixed oxide.

4. The method for producing fatty acid alkyl esters and/or glycerin according to claim 1,
wherein the metallic element belonging to the Group 12 is zinc element.

5. The method for producing fatty acid alkyl esters and/or glycerin according to claim 1,
wherein the catalyst is an oxide containing a metallic element belonging to the Group 12, and Ti or Zr.

6. The method for producing fatty acid alkyl esters and/or glycerin according to claim 1,
wherein the oxide contains ZnₓMOₙ in the formula, M represents Ti and/or Zr; x is a number of 0.05 or more and 10 or less; and n is a number determined such that ZnₓMOₙ is electrically neutral.

7. The method for producing fatty acid alkyl esters and/or glycerin according to claim 6,
wherein the oxide contains at least one selected from the group consisting of ZnZrO₃, ZnTiO₃, Zn₂TiO₄, Zn_{2/3}TiO_{8/3}, and Zn₄TiO₆.

8. The method for producing fatty acid alkyl esters and/or glycerin according to claim 5,
wherein the oxide is cubic.

9. The method for producing fatty acid alkyl esters and/or glycerin according to claim 1,
wherein the catalyst contains zirconium oxide having a monoclinic structure.

10. The method for producing fatty acid alkyl esters and/or glycerin according to claim 1,
wherein the catalyst contains a metal oxide having a triclinic structure as an active component.

11. The method for producing fatty acid alkyl esters and/or glycerin according to claim 1,
wherein the catalyst contains an oxide of at least one metallic element selected from the group consisting of metallic elements belonging to the Groups 4, 13, and 14.

12. A catalyst for producing fatty acid alkyl esters and/or glycerin, wherein the catalyst is used in the method for producing fatty acid alkyl esters and/or glycerin according to claim 1.

13. The method for producing fatty acid alkyl esters and/or glycerin according to claims 2,
wherein the metallic element belonging to the Group 12 is zinc element.

14. The method for producing fatty acid alkyl esters and/or glycerin according to claims 3,
wherein the metallic element belonging to the Group 12 is zinc element.

15. The method for producing fatty acid alkyl esters and/or glycerin according to claim 3,
wherein the catalyst is an oxide containing a metallic element belonging to the Group 12, and Ti or Zr.

16. The method for producing fatty acid alkyl esters and/or glycerin according to claim 4,
wherein the catalyst is an oxide containing a metallic element belonging to the Group 12, and Ti or Zr.

17. The method for producing fatty acid alkyl esters and/or glycerin according to claim 3,
wherein the oxide contains ZnₓMOₙ (in the formula, M represents Ti and/or Zr; x is a number of 0.05 or more and 10 or less; and n is a number determined such that ZnₓMOₙ is electrically neutral).

18. The method for producing fatty acid alkyl esters and/or glycerin according to claim 4,
wherein the oxide contains ZnₓMOₙ in the formula, M represents Ti and/or Zr; x is a number of 0.05 or more and 10 or less; and n is a number determined such that ZnₓMOₙ is electrically neutral.

19. The method for producing fatty acid alkyl esters and/or glycerin according to claim 5,
wherein the oxide contains ZnₓMOₙ in the formula, M represents Ti and/or Zr; x is a number of 0.05 or more and 10 or less; and n is a number determined such that ZnₓMOₙ is electrically neutral.

20. The method for producing fatty acid alkyl esters and/or glycerin according to claim 6,
wherein the oxide is cubic.
